(19) United States
(12) Patent Application Publication
(10) Pub. No.: US 2024/0307012 A1
(43) Pub. Date: Sep. 19, 2024

(54) RADIOGRAPHIC IMAGING SUPPORT APPARATUS, RADIOGRAPHIC IMAGING SUPPORT METHOD, AND RECORDING MEDIUM

(71) Applicant: Konica Minolta, Inc., Tokyo (JP)

(72) Inventors: Satoshi HASEGAWA, Tokyo (JP); Naoki HAYASHI, Tokyo (JP); Akira HIROSHIGE, Tokyo (JP); Kenichi YANAGISAWA, Tokyo (JP)

(21) Appl. No.: 18/603,788

(22) Filed: Mar. 13, 2024

(30) Foreign Application Priority Data
Mar. 16, 2023 (JP) 2023-041460

(51) Int. Cl. A61B 6/00; A61B 6/42; A61B 6/46; G01T 1/20

(52) U.S. Cl. CPC A61B 6/4405; A61B 6/4291; A61B 6/469; A61B 6/5294; G01T 1/2018

(57) ABSTRACT

A radiographic imaging support apparatus includes a hardware processor. The hardware processor performs notification of information on imaging equipment to be used by a photographer at a destination. The imaging equipment is determined based on examination information on a subject to be subjected to radiographic imaging by a movable radiographic imaging apparatus.

### REFERENCE TO RELATED APPLICATIONS

The entire disclosure of Japanese Patent Application No. 2023-041460, filed on Mar. 16, 2023, including description, claims, drawings and abstract is incorporated herein by reference in its entirety.

### BACKGROUND OF THE INVENTION

### Technical Field

The present invention relates to a radiographic imaging support apparatus, a radiographic imaging support method, and a recording medium.

### Description of Related Art

Conventionally, when a user such as a radiographer visits a patient at a ward or the like of a medical facility such as a hospital, the user captures a radiation (X-ray) image of the patient at the visit location. In this case, the user captures an image with a movable radiographic imaging apparatus (medical cart) including a flat panel detector (FPD). At the visit location, one patient may be the imaging target, or a plurality of patients may be continuously imaged. The user needs to go to the visit location in preparation for the imaging situation at the visit location.

For example, when the X-ray imaging apparatus described in Japanese Unexamined Patent Publication No. 2012-50620 is set to perform imaging with a medical cart, the X-ray imaging apparatus determines, regardless of imaging conditions, to use one of a plurality of wireless sensors (FPDs) that has the highest remaining battery level.

Meanwhile, in the case of imaging at the visit location, the user generally goes to a standby place where an FPD and a medical cart are present. Next, the user selects an FPD and a medical cart to be used for imaging. Next, the user takes the selected FPD and medical cart to the visit location and performs imaging.

Such imaging at the visit location has the following problem as compared with general imaging performed in an imaging room.

Specifically, since a patient who is the imaging target is not present at the time of selecting an FPD to be used for imaging, the user cannot directly confirm the patient as in general imaging and cannot determine the size of the FPD to be taken to the visit location on the basis of the physique or the like of the patient.

Since it is assumed that the patient cannot move in imaging at the visit location, a fixing jig for fixing the patient, an assisting tool, and/or the like may be needed. However, when the user selects a fixing jig, an assisting tool, and/or the like to be used for imaging, the patient to be imaged is not present on the spot. Hence, the user cannot directly check the patient as in general imaging, and cannot determine a fixing jig, an assisting tool, and/or condition of the patient.

Since an environment such as a hospital ward to be visited is unknown when the user selects a fixing jig, an assisting tool, and/or the like to be used for imaging, the user cannot directly confirm the environment as in general imaging and cannot determine a fixing jig, an assisting tool, and/or the like to be taken to the visit location on the basis of the environment of the visit location.

In a case where a terminal such as an electronic medical record or radiology information systems (RIS) is not nearby at the visit location and communication is weak in a communication environment such as a hospital ward as the visit location, the user may not be able to check imaging equipment necessary for imaging at the visit location. The imaging equipment includes an FPD, a medical cart, a fixing jig, and an assisting tool.

Although selection from such pieces of imaging equipment can be performed by an experienced radiographer on the basis of an empirical rule, it is the minimum selection and is hardly said to be the optimum selection. Furthermore, it is more difficult for an inexperienced radiographer.

### SUMMARY OF THE INVENTION

It is required to solve the above-described problem and allow a user to take imaging equipment suitable for imaging at a visit location to the visit location.

In the invention described in Japanese Unexamined Patent Publication No. 2012-50620, for imaging with a medical cart, an FPD to be used for imaging regardless of imaging conditions is selected. Therefore, in the invention, there is a possibility that imaging equipment including an FPD suitable for imaging at the visit location cannot be selected.

Objects of the present invention include providing a radiographic imaging support apparatus, a radiographic imaging support method and a recording medium by which imaging equipment suitable for imaging at a visit location can be prepared before going to the visit location.

To achieve at least one of the abovementioned objects, according to an aspect of the present invention, a radiographic imaging support apparatus reflecting one aspect of the present invention includes a hardware processor that performs notification of information on imaging equipment to be used by a photographer at a destination, the imaging equipment being determined based on examination information on a subject to be subjected to radiographic imaging by a movable radiographic imaging apparatus.

According to an aspect of the present invention, a radiographic imaging support method reflecting one aspect of the present invention includes performing notification of information on imaging equipment to be used by a photographer at a destination, the imaging equipment being determined based on examination information on a subject to be subjected to radiographic imaging by a movable radiographic imaging apparatus.

According to an aspect of the present invention, a non-transitory computer-readable recording medium reflecting one aspect of the present invention stores a program that causes a hardware processor of a radiographic imaging support apparatus to perform notification of information on imaging equipment to be used by a photographer at a destination, the imaging equipment being determined based on examination information on a subject to be subjected to radiographic imaging by a movable radiographic imaging apparatus.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 is a schematic configurational view illustrating a radiographic imaging system of the present embodiment;

FIG. 2 is a block diagram illustrating a configuration of a medical cart;

FIG. 3 is a flowchart illustrating a recommended equipment information display process;

FIG. 4 shows an example of a display screen for displaying examination information;

FIG. 5 shows an example of a display screen for displaying recommended equipment information;

FIG. 6 shows an example of a recommended equipment information screen for displaying the recommended equipment information;

FIG. 7 shows an example of the display screen for displaying the recommended equipment information;

FIG. 8 shows an example of the display screen for displaying the recommended equipment information;

FIG. 9 shows an example of the display screen for displaying the recommended equipment information;

FIG. 10 shows an example of the display screen for displaying the recommended equipment information;

FIG. 11 shows an example of the recommended equipment information screen for displaying the recommended equipment information;

FIG. 12 shows an example of a reason screen for displaying recommendation reason information; and

FIG. 13 is a schematic configuration diagram illustrating a radiation system of a modification example.

### DETAILED DESCRIPTION

Hereinafter, one or more embodiments of the present invention will be described with reference to the drawings. However, the scope of the invention is not limited to the disclosed embodiments.

### 1. Configuration of Radiographic Imaging System

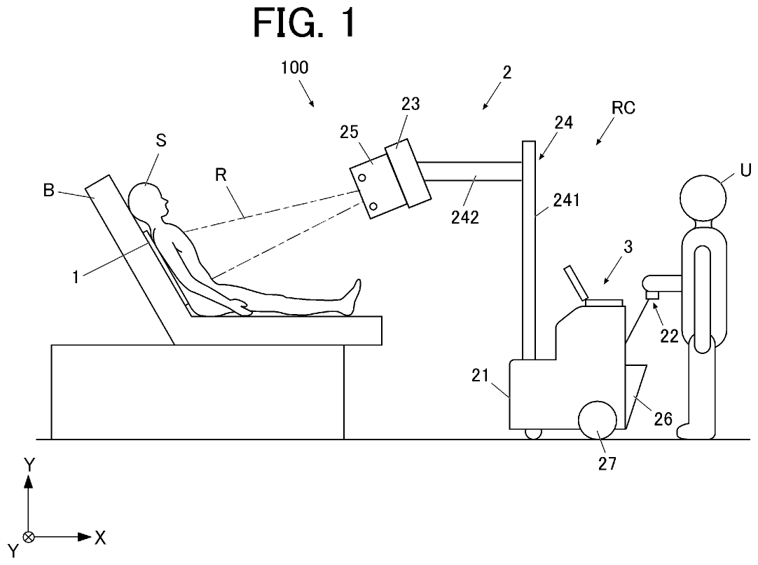

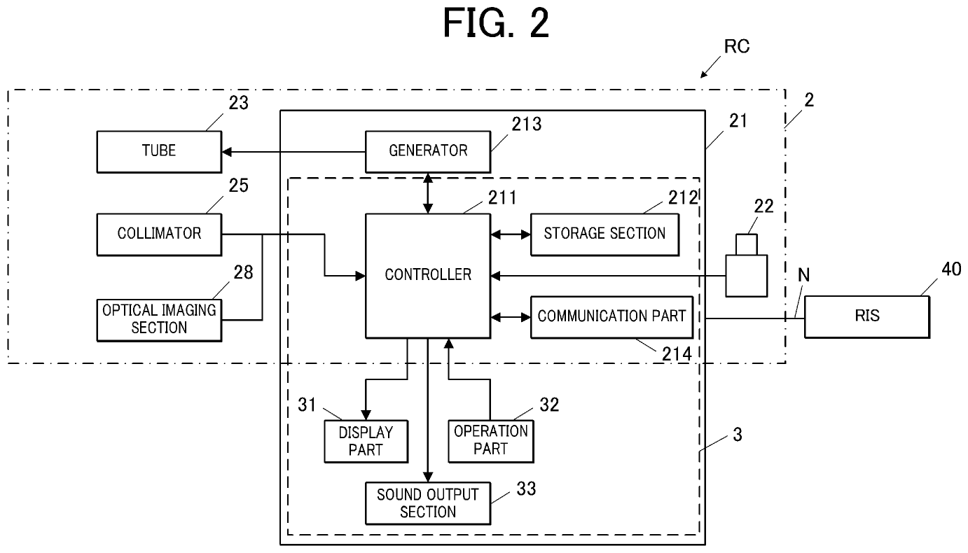

First, the apparatus configuration according to the present embodiment will be described with reference to FIGS. 1 and 2. FIG. 1 is a schematic configuration diagram illustrating a radiographic imaging system 100. FIG. 2 is a block diagram illustrating a configuration of a medical cart RC.

The radiographic imaging system 100 illustrated in FIG. 1 is provided in a medical facility such as a hospital.

A user U such as a radiographer of a medical facility moves to a visit location such as a ward or an operating room where a patient is present together with the radiographic imaging system 100 at the time of round visit of the patient as a round visit target, and performs radiographic imaging of the patient. An example will be described in which a visit location is a hospital ward, a bed B is installed in the hospital ward, and radiographic imaging is performed in a state in which a subject S who is a patient as a subject is in a lying position on the bed B whose back is raised.

The radiographic imaging system 100 includes an FPD 1, which is a radiographic imaging apparatus, and a medical cart RC, which is a movable radiographic imaging apparatus.

The FPD 1 and the medical cart RC are communicable with each other, for example, via wireless communication.

The FPD 1 is an apparatus that generates radiographic image data corresponding to radiation R emitted from a radiation generation apparatus 2.

The FPD 1 is formed in a panel shape and can be carried.

The user U can use the FPD 1 by mounting it on the imaging table. The user U can use the FPD 1 horizontally placed between the subject S lying on the bed B and the bed B. The user U can use the FPD1 standing between the backrest and the subject S, who is in a sitting posture in the raised bed B or wheelchair, as shown in FIG. 1.

The FPD 1 includes, for example, scintillators, photo diodes (PD), and thin film transistor (TFT) switches (all not shown). The scintillator converts the incident radiation R into light. The PDs are arranged in a matrix corresponding to the pixels. The TFT switch is arranged corresponding to each PD.

The incident radiation R is converted into light by the scintillator, and the light is incident on the PD and is accumulated as charges for each pixel. The charges accumulated in the PDs flow out through the TFT switches and the signal lines, are amplified and A/D converted, and are output as radiographic image data to the medical cart RC.

The FPD 1 may be the above-described indirect conversion type, or may be a direct conversion type that directly converts the radiations R into electrical signals.

As illustrated in FIG. 2, the medical cart RC is communicable with the RIS 40 via the communication network N. The medical cart RC may be able to communicate with an electronic medical record system, a console included in another radiographic imaging system, another medical cart, a picture archiving and communication system (PACS), another examination apparatus, a bed system, and the like via the communication network N.

The communication network N is wireless, but may be wired.

The medical cart RC includes a radiation generation apparatus 2 and a console 3.

The radiation generation apparatus 2 includes a generator main body 21, an irradiation instruction switch 22, a tube 23, a tube support portion 24, a collimator 25, and an FPD storage section 26 (storage). The radiation generation apparatus 2 can be moved by wheels 27 provided on the housing of the generator main body 21.

The irradiation instruction switch 22 outputs an operation signal to the generator main body 21 in response to being operated (pressed) by the user U. FIG. 1 illustrates a state in which the irradiation instruction switch 22 is connected to the generator main body 21 by wire. However, the irradiation instruction switch 22 and the generator main body 21 may be wirelessly connected to each other.

When the irradiation instruction switch 22 is operated, the tube 23 generates the radiation R (X-rays or the like) of a dose corresponding to a preset imaging condition in a mode corresponding to the imaging condition. Next, the tube 23 emits the generated radiation R from an irradiation port.

The tube support portion 24 is an arm that supports the tube 23.

The tube support portion 24 includes a support portion 241 extending from the generator main body 21 to an upper distal end, and a support portion 242 extending forward from an upper portion of the support portion 241. A distal end portion of the support portion 242 supports the tube 23.

The tube support portion 24 can move the tube 23 in an X-axis direction, a Y-axis direction orthogonal to the X-axis, and a Z-axis direction orthogonal to the X-axis and the Y-axis by having a joint mechanism (not shown). The X-axis direction in FIG. 1 is the right-left direction of FIG. 1, that is, the front-rear direction of the radiation generation apparatus 2. The Y-axis direction in FIG. 1 is a direction orthogonal to the sheet surface of FIG. 1, that is, the width direction of the radiation generation apparatus 2. The Z-axis direction in FIG. 1 is an up-down direction in FIG. 1, that is, a vertical direction.

The tube support portion 24 can change the direction of the irradiation port of the radiation R by rotating the tube 23 around rotation axes parallel to the X axis, the Y axis, and the Z axis by the joint mechanism (not illustrated).

The collimator 25 is attached to the irradiation port of the tube 23 and narrows the radiation R so that the irradiation field of the radiation R emitted from the irradiation port has a preset rectangular shape.

The collimator 25 includes a lamp button (not illustrated). The collimator 25 irradiates a range to be an irradiation field of the radiation R with visible light when a lamp button is operated by a user.

The FPD storage section 26 is provided on a side surface of the generator main body 21, and stores the FPD 1 when not in use. The FPD storage section 26 can store one or a plurality of FPDs 1.

A connector (not illustrated) is provided in the FPD storage section 26, and may be connected to a connector (not illustrated) included in the FPD 1 when the FPD 1 is stored.

The console 3 includes a personal computer (PC), a portable terminal, or a dedicated apparatus, and is mounted on the radiation generation apparatus 2.

The console 3 can set an imaging condition in at least one of the FPD 1 and the radiation generation apparatus 2.

The imaging conditions include a tube voltage, a tube current and an irradiation time or a current-time product (mAs value), an imaging site, an imaging direction, and the like. In the case of dynamic imaging, the imaging conditions further include a frame rate.

The imaging conditions are based on examination information obtained from an external apparatus such as the RIS 40, or on operations made by the user U on the operation part 32.

The examination information is information about radiographic imaging that the clinician requests the user to perform.

The console 3 can acquire radiographic image data generated by the FPD 1, store the radiographic image data in itself or transmit the radiographic image data to another external apparatus (e.g., PACS).

Sitting position imaging, which is radiographic imaging using the radiographic imaging system 100 configured as described above, is performed as follows.

The user U places the radiographic imaging system 100 near the subject S, for example, on the side of the bed B. Next, the user U causes the subject S to take a sitting posture. The user U appropriately adjusts the angle of the backrest portion of the bed B if the subject S is sitting on an instrument such as the bed B whose angle is adjustable. Next, the user U adjusts the approximate position and direction of the tube 23 so that the irradiation port of the tube 23 is directed toward an imaging site of the subject S. Next, the user U takes out the FPD 1 from the FPD storage section 26 and places the FPD 1 between the back of the subject S and the backrest. Next, the user U finely adjusts the direction of the tube 23 and the irradiation field so that the emission axes of the radiation R are orthogonal to the 15 radiation incident surface of the FPD 1. Next, the user U performs radiographic imaging. That is, the user U causes the tube 23 to irradiate an imaging site of the subject S with the radiation R, and causes the FPD 1 to generate radiographic image data of a still or dynamic image of the diagnostic site.

In the present embodiment, the generator main body 21 and the console 3 are configured to be stored in one housing by being integrated with each other, but may be separate bodies.

The radiation generation apparatus 2 may be movable by means other than the wheels 27. For example, the radiation generation apparatus 2 may be light enough to be carried by a person or mounted on a commercially available cart or the like. The radiation generation apparatus 2 may have a smooth bottom surface to be slidable on a floor surface.

Of the radiographic imaging system 100, the radiation generation apparatus 2 may be installed in a room of a medical facility.

Next, the internal configurations of the radiation generation apparatus 2 and the console 3 of the medical cart RC will be described with reference to FIG. 2.

The radiation generation apparatus 2 further includes an optical imaging section 28.

The generator main body 21 includes a controller 211 (hardware processor), a storage section 212, a generator 213, and a communication part 214.

The components of the radiation generation apparatus 2 except for the tube 23 are communicably connected.

The optical imaging section 28 includes an optical system such as a lens and an imaging element such as a charge coupled device (CCD) or a complementary metal oxide semiconductor (CMOS).

In the present embodiment, the optical imaging section 28 is provided in a housing of the collimator 25. The optical imaging section 28 may be provided in a housing of the tube 23 or in the tube support portion 24.

Under the control of the controller 211, the optical imaging section 28 optically images the subject S as the object with visible light to generate optical image data, and outputs the optical image data to the controller 211 and the like. The optical imaging section 28 optically images the subject S to generate optical image data of a still image or a dynamic image (such as a live image).

The controller 211 includes a CPU and a RAM. The CPU reads various programs stored in the storage section 212 and loads the programs in the RAM. The CPU executes various kinds of processing in cooperation with the programs, and controls the components of the radiation generation apparatus 2 and the console 3.

The storage section 212 includes a nonvolatile memory and an HDD.

The storage section 212 stores various programs to be executed by the controller 211 and various types of data such as parameters and files necessary for executing the programs.

The storage section 212 stores a recommended equipment information display program for executing a recommended equipment information display process to be described later.

In response to receipt of the imaging instruction signal from the controller 211, the generator 213 applies a voltage corresponding to the preset imaging conditions to the tube 23 and applies a current corresponding to the imaging conditions to the tube 23.

The communication part 214 includes a communication module and the like.

The communication part 214 can transmit and receive various signals, various data and the like to and from the wirelessly connected FPD 1 and/or an external apparatus such as the RIS 40 connected via the communication network N.

The console 3 includes a controller, a storage section, a communication part, a display part 31, an operation part 32, and a sound output section 33. The controller 211, the storage section 212, and the communication part 214 of the radiation generation apparatus 2 serve as the controller, the storage section, and the communication part, respectively, of the console 3. The console 3 may include a dedicated controller, a dedicated storage section, and a dedicated communication part.

The display part 31 includes a liquid crystal display (LCD), an electro-luminescence (EL) display, or the like.

The display part 31 displays various kinds of information according to display information input from the controller 211.

The operation part 32 includes, for example, a keyboard having various keys, a pointing device for inputting position information, and a touch screen integrally formed on the display screen of the display part 31.

The operation part 32 receives a user's operation input, and outputs the operation information to the controller 211.

The sound output section 33 includes an amplifier, a speaker, and the like and outputs sound in accordance with sound information input from the controller 211.

### 2. Operation of Radiographic Imaging System

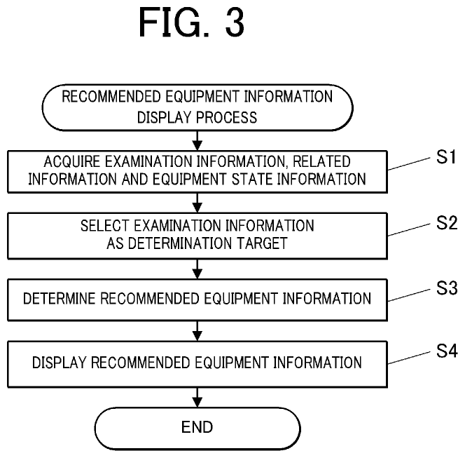

Next, operation of the radiographic imaging system 100 will be described with reference to FIG. 3.

A user, such as a radiographer, goes to a standby place where the FPD 1 and the medical cart RC are present before going to a visit location where a subject to be imaged is present. Next, in the medical cart RC, the controller 211 executes a recommended equipment information display process shown in FIG. 3 in accordance with the recommended equipment information display program stored in the storage section 212.

By executing the recommended equipment information display process, the controller 211 determines recommended equipment information indicating the imaging equipment recommended as the imaging equipment to be used for radiographic imaging to be performed at the visit location. Next, the controller 211 notifies the user of the recommended equipment information by displaying the determined recommended equipment information on the display part 31.

That is, the controller 211 performs notification of information about the imaging equipment to be used by the photographer at the destination, which is determined based on the examination information about the subject to be radiographed by the medical cart RC as the movable radiographic imaging apparatus. The controller 211 functions as a notification section. In this case, the console 3 is the radiographic imaging support apparatus.

The imaging equipment includes the FPD 1, a grid detachably attached to the FPD 1, a fixing jig for fixing the subject, and a assisting tool.

### (Recommended Equipment Information Display Process)

The controller 211 acquires examination information, related information related to the examination information, and equipment state information that is information on the state of the imaging equipment (Step S1).

The examination information acquired in Step S1 includes an examination ID, an examination date and time, a requesting department, visit location, imaging information, and the like.

The visit location is a place where radiographic imaging is performed, and for example, is a hospital ward, an intensive care unit, an operating room, a general imaging room, or the like.

The imaging information includes the imaging site, the imaging direction, the size of the FPD 1 corresponding to the imaging, the resolution, information on the type of imaging, the irradiation conditions, the radiation dose indicator in imaging this time, the necessity/unnecessity of the grid, the type of the grid, and the like.

The information on the type of imaging is still imaging, dynamic imaging, fluoroscopic imaging, or the like.

A series of images obtained by dynamic imaging is referred to as a dynamic image. Each of images constituting a dynamic image is referred to as a frame image.

The dynamic imaging includes video shooting, but does not include still imaging while a moving image is displayed. The dynamic image includes a moving image, but does not include an image obtained by still imaging while a moving image is displayed.

The irradiation conditions include an mAs value, a tube voltage, a frame rate, an irradiation time, and the like.

The related information acquired in Step S1 includes patient information, past imaging information, statistical information, and the like.

The patient information is information on a patient as a subject who is a target of radiographic imaging. Specifically, the patient information includes a patient ID, a name, a gender, a pregnancy state, an age, a height, a weight, medical precautions, a medical history, a residence in a facility such as a hospital ward, and a state of the patient. The state of the patient is, for example, that the patient is in a hunched posture, that the patient is unlikely to lie on its side, that the patient has a tube for intravenous drip or the like, or the like.

The past imaging information is imaging information about radiographic imaging performed in the past. To be specific, the past imaging information includes information on an imaging site, an imaging direction, the size of the FPD 1 corresponding to imaging, resolution, information on the type of imaging, irradiation conditions, a trimming size at the time of imaging in the past, precautions at the time of imaging in the past, the presence/absence of a grid, the type of a grid, the presence/absence of application of image processing, and an imaging location.

The precautions at the time of imaging in the past include, for example, the number of radiographers who performed imaging last time.

The image processing is, for example, scattered radiation correction.

The imaging location is, for example, a hospital ward, an intensive care unit, an operating room, or a general imaging room.

The statistical information includes the frequency of use of a combination of the medical cart RC and the FPD 1 in radiographic imaging, the frequency of use of a combination of imaging information and the FPD 1 in radiographic imaging, the frequency of use of a combination of a case of a subject and the FPD 1 in radiographic imaging, and the frequency of use of a combination of radiographer information and a fixing jog and/or an assisting tool in radiographic imaging. The radiographer information is information on a radiographer who performs radiographic imaging.

The equipment state information acquired in Step S1 includes location information of the FPD 1, remaining battery level information of the FPD 1, the type of the FPD 1, location information of the medical cart RC, charging state information of the medical cart RC, the number of FPDs 1 storable in the medical cart RC, the date of the last imaging by the medical cart RC, the imaging interval by the medical cart RC, and radiographer information.

In Step S1, for example, the controller 211 acquires the examination information and the patient information from the RIS 40 via the communication part 214 every predetermined time.

When an instruction to update the examination information is input by the user via the operation part 32, the controller 211 may acquire the examination information and the patient information from the RIS 40 via the communication part 214.

The controller 211 may acquire the examination information and the patient information as follows. Specifically, the controller 211 receives input of the patient information by the user via the operation part 32. Next, the controller 211 transmits the received patient information to the RIS 40. The RIS 40 transmits the examination information based on the received patient information to the medical cart RC. Next, the controller 211 receives the examination information from the RIS 40.

If the examination information is stored in the storage section 212, the controller 211 may acquire the examination information as follows. Specifically, the controller 211 acquires the examination information from the storage section 212 on the basis of the patient information for which input has been received via the operation part 32.

If the patient information, imaging information, past imaging information, statistical information, and equipment state information are stored in the storage section 212, the controller 211 acquires these pieces of information from the storage section 212.

In a case where the patient information, the imaging information, the past imaging information, the statistical information, and the equipment state information are stored in a storage section included in another radiographic imaging system, the controller 211 acquires these pieces of information from the other radiographic imaging system.

The controller 211 may acquire the equipment state information from another medical cart RC.

The controller 211 may acquire the patient information, the imaging information, and the equipment state information from an apparatus such as a PACS, another examination apparatus, or a bed system.

The controller 211 may acquire the patient information, the imaging information, and the equipment state information based on the optical image data generated by the optical imaging section 28.

In addition to the patient information, the controller 211 may acquire the examination information and the equipment state information by user's input via the operation part 32.

The controller 211 may acquire the patient information from a digital image and communications in medicine (DICOM) tag.

Next, the controller 211 selects examination information for which the recommended equipment information indicating recommended imaging equipment for radiographic imaging to be performed at the visit location is to be determined (Step S2).

For example, the controller 211 sets all pieces of the examination information acquired from the RIS 40 in Step S1 as a determination target. The examination information transmitted from the RIS 40 to the medical cart RC is examination information for round-visit imaging to be performed by the medical cart RC. Therefore, in this case, the controller 211 sets the examination information for round-visit imaging to be performed by its own apparatus, namely, the medical cart RC, as the determination target.

The controller 211 may select the examination information as the determination target as follows. Specifically, the controller 211 causes the display part 31 to display a list of pieces of the examination information as candidates for the determination target. The candidates for the determination target include examination information for round-visit imaging to be performed by the own medical cart RC, examination information for round-visit imaging to be performed by another medical cart RC, and the like. Next, the controller 211 receives, from the user, a selection input of a piece of the examination information as the determination target among the displayed pieces of the examination information, via the operation part 32. Next, the controller 211 sets the examination information selected by the user as the examination information as the determination target.

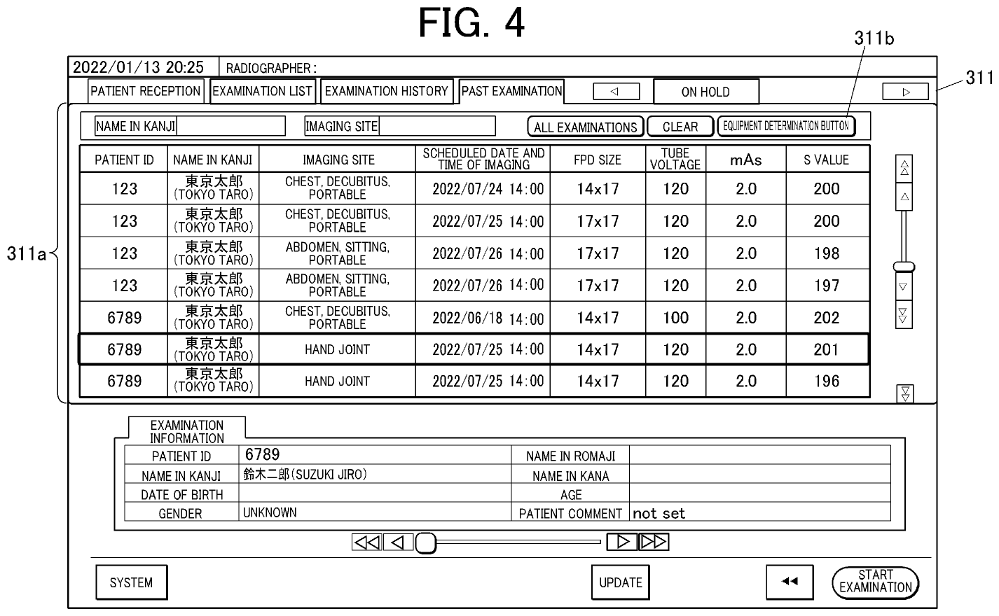

FIG. 4 shows an example of a display screen 311 displayed on the display part 31.

The controller 211 displays, in a list 311a in the display screen 311, pieces of the examination information that are candidates for the determination target and pieces of related information related to the pieces of the examination information. The controller 211 displays, in the list 311a, for example, the patient ID, patient name, imaging site, scheduled date and time of imaging, size of the FPD 1 corresponding to imaging, imaging conditions, and the like. The imaging conditions include a tube voltage, an mAs value, an S value, and the like. The S value is sensitivity corresponding to a radiation dose at the time of radiographic imaging.

The controller 211 receives, from the user via the operation part 32, an input of selection of a piece of the examination information displayed in the list 311a.

The controller 211 may display, in the list 311a, a check box or the like to show a selection input from the user.

The controller 211 may provide a tab for displaying only the examination information for round-visit imaging on the display screen 311.

The controller 211 may display the examination information selected by the user in another tab in the display screen 311 or in a screen other than the display screen 311, which is dialog display or the like.

When the controller 211 receives an input by a press on a equipment determination button 311b in the display screen 311 from the user in a state in which the examination information as the determination target is selected, the controller 211 moves the process to Step S3.

The controller 211 may select the examination information as the determination target as follows. Specifically, the controller 211 selects the examination information as the determination target on the basis of selection conditions preset for each facility, each user, or the like.

The selection conditions include, for example, examination information for not-yet imaging, examination information with a patient in a specific ward being the imaging target, examination information with the patient imaged in a focused predetermined examination being the imaging target, or the like.

The controller 211 may select examination information on one subject as the determination target, or may select examination information on a plurality of subjects as the determination target.

The controller 211 may select, in Step S2, the determination target from pieces of the examination information for round-visit imaging only.

In Step S2, the controller 211 may select the determination target from pieces of the examination information for general imaging, dynamic imaging, and round-visit imaging.

Next, the controller 211 determines recommended equipment information corresponding to the examination information selected in Step S2 as the determination target, on the basis of the examination information, the related information, and the equipment state information (Step S3).

The recommended equipment information of the present embodiment includes a recommended FPD 1 size.

A specific method for determining the recommended FPD 1 size will be described below.

For example, the controller 211 determines, as the recommended FPD 1 size, the maximum size among the sizes of the FPD 1 based on the imaging information included in a plurality of pieces of the examination information as the determination target.

The controller 211 determines the order of recommendation of FPD 1 sizes based on a preset order of priority. For example, the controller 211 sets the order of recommendation for the size of the FPD 1 based on the imaging information included in the examination information as the determination target and the size of the FPD 1 used when the same patient as the patient corresponding to the examination information was imaged in the past.

The controller 211 estimates the physique of the patient from the patient information in radiographic imaging of the patient in a predetermined period in the past, the patient corresponding to the examination information as the determination target. Next, the controller 211 determines the size of the FPD 1 corresponding to the estimated physique as the recommended FPD 1 size. The predetermined period is, for example, one year.

The controller 211 determines, as the recommended FPD 1 size, the size of the FPD 1 based on the height and weight of the patient related to the examination information as the determination target.

A case where the size of the FPD 1 included in the examination information as the determination target and the size of the FPD 1 included in past imaging information related to the examination information do not match each other will be described. In this case, the controller 211 determines the size of the FPD 1 included in the past imaging information as the recommended FPD 1 size.

The controller 211 determines, as the recommended FPD 1 size, the size of the FPD 1 based on the trimming size included in the past imaging information related to the examination information as the determination target. To be specific, the controller 211 determines, as the recommended FPD 1 size, the size of the FPD 1 that can include the trimming size in the past radiographic imaging.

If the gender of the radiographer included in the radiographer information is female, the controller 211 determines a relatively small size of the FPD 1 as the recommended FPD 1 size.

The recommended equipment information of the present embodiment includes the number of types having the recommended FPD 1 size.

A specific method for determining the number of types having the recommended FPD 1 size will be described below.

For example, the controller 211 determines the number of types having the recommended FPD 1 size according to the size of the FPD storage section 26. In this case, the equipment state information includes the size of the FPD storage section 26.

The recommended equipment information of the present embodiment includes type information of a recommended FPD 1.

A specific method of determining the type information of the recommended FPD 1 will be described below.

For example, if the information on the type of imaging included in the examination information as the determination target is dynamic imaging, the controller 211 determines the type of the FPD 1 capable of performing dynamic imaging as the recommended FPD 1 type.

If the information on a frame rate is included in the irradiation conditions included in the examination information as the determination target, the controller 211 determines that the type of imaging is dynamic imaging. Next, the controller 211 determines the type of the FPD 1 capable of performing dynamic imaging as the recommended FPD 1 type.

If the value of the dose index included in the examination information as the determination target is larger than a predetermined threshold, the controller 211 determines that the type of imaging is dynamic imaging. Next, the controller 211 determines the type of the FPD 1 capable of performing dynamic imaging as the recommended FPD 1 type.

If the imaging information included in the examination information as the determination target indicates that the resolution is high definition, the controller 211 determines the type of the FPD 1 capable of performing imaging with the reading resolution being high definition as the recommended FPD 1 type.

The controller 211 determines a case as the imaging target on the basis of the requesting department and the imaging site included in the examination information as the determination target. Next, the controller 211 determines, as the recommended FPD 1 type, the type of the FPD 1 capable of performing imaging with the resolution suitable for the determination result.

The controller 211 determines, as the recommended FPD 1 type, the type of the FPD 1 based on the imaging site included in the past imaging information related to the examination information as the determination target. To be specific, the controller 211 determines, as the recommended FPD 1 type, the type of the FPD 1 used in the past radiographic imaging by which the same site as the imaging site of this time was imaged.

The controller 211 determines, as the recommended FPD 1 type, the type of the FPD 1 based on the information on the type of imaging included in the past imaging information related to the examination information as the determination target. To be specific, the controller 211 determines, as the recommended FPD 1 type, the type of the FPD 1 used in the past radiographic imaging of the same type as the type of imaging of this time.

The controller 211 determines, as the recommended type FPD 1 type, the type of the FPD 1 based on the irradiation conditions included in the past imaging information related to the examination information as the determination target. To be specific, the controller 211 determines, as the recommended FPD 1 type, the type of the FPD 1 used in the past radiographic imaging under the same irradiation conditions as the irradiation conditions of this time.

The controller 211 determines, as the recommended FPD 1 type, the type of the FPD 1 based on the precautions taken at the time of past imaging included in the past imaging information related to the examination information as the determination target.

A case in which the resolution included in the examination information as the determination target does not match the resolution included in the past imaging information related to the examination information will be described. In this case, the controller 211 determines, as the recommended FPD 1 type, the type of the FPD 1 capable of performing imaging with the resolution included in the past imaging information.

The controller 211 determines whether the FPD 1 type determined as described above matches the FPD 1 type included in the equipment state information. If these FPD 1 types match, the controller 211 may determine the determined FPD 1 type as the recommended FPD 1 type.

The recommended equipment information of the present embodiment includes identification information of a recommended FPD 1.

A specific method of determining the identification information of the recommended FPD 1 will be described below.

For example, a case where there is a plurality of FPDs 1 having the recommended FPD 1 size will be described.

In this case, the controller 211 determines, based on the statistical information, an FPD 1 with a high use frequency for the imaging information included in the examination information as the determination target as the recommended FPD 1.

The controller 211 determines, based on the statistical information, an FPD 1 that is frequently used in combination with the medical cart RC as the recommended FPD 1.

Based on the statistical information, the controller 211 determines, as the recommended FPD 1, an FPD 1 that is frequently used in combination with the case of the subject corresponding to the examination information as the determination target.

The controller 211 determines to exclude, from the recommended FPD 1, an FPD 1 whose location included in the equipment state information is separated by a predetermined distance or more from the visit location included in the examination information that is the determination target.

The controller 211 determines to exclude, from the recommended FPD 1, an FPD 1 for which the remaining battery level of the FPD 1 included in the equipment state information is equal to or lower than a predetermined level.

The controller 211 determines to exclude, from the recommended FPD 1, the FPD 1 in which the remaining battery level of the FPD 1 included in the equipment state information is lower than the remaining level at which the imaging of the examination information as determination target can be performed.

The recommended equipment information of the present embodiment includes type information of a recommended grid.

A specific method of determining the type information of the recommended grid will be described below.

For example, the controller 211 determines a grid type corresponding to the size of the FPD 1 based on the imaging information included in the examination information as the determination target as the recommended grid type.

The controller 211 determines the type of grid corresponding to the imaging site included in the examination information as the determination target as the recommended type of grid. For example, if the imaging site is the chest, the controller 211 determines a grid having a lattice ratio of 10:1 as a recommended grid. In a case where the imaging site is the abdomen, the controller 211 determines the grid with a lattice ratio of 8:1 as the recommended grid.

If the information about the type of imaging included in the examination information as the determination target is dynamic imaging, the controller 211 determines a grid for dynamic imaging as the recommended grid.

The controller 211 determines the type of grid corresponding to the visit location included in the examination information as the determination target as the recommended type of grid. For example, in a case where the visit location is an operation room or an emergency ward, the controller 211 determines a type of grid corresponding to an FPD 1 that can be used on an operation table, a custom-made bed for emergency, or the like as the recommended type of grid.

The controller 211 determines, as a recommended grid type, a grid type based on the past imaging information related to the examination information as the determination target. For example, a case in which scattered radiation correction was used in the previously performed radiographic imaging and there was no grid will be described. In this case, the controller 211 determines that the grid is not used as the type of the recommended grid.

The recommended equipment information of the present embodiment includes type information of the recommended fixing jig and assisting tool.

A specific method of determining the type information of the recommended fixing jig and assisting tool will be described below.

For example, the controller 211 determines, as the recommended types of the fixing jig and the assisting tool, the types of the fixing jig and the assisting tool corresponding to the imaging site included in the examination information as the determination target. For example, a case will be described in which the imaging site is a side of the chest or the like, and the subject needs to be moved and fixed at the time of imaging. In this case, the controller 211 determines the types of fixing jig and assisting tool that are necessary for the operation of moving and fixing the subject as the recommended types of fixing jig and assisting tool.

The controller 211 determines the types of the fixing jig and the assisting tool based on the imaging site included in the past imaging information related to the examination information as the determination target as the recommended types of the fixing jig and the assisting tool. Specifically, the controller 211 determines the types of the fixing jig and the assisting tool used in the past radiographic imaging in which the same imaging site as the imaging site of this time was imaged, as the recommended types of the fixing jig and the assisting tool.

The controller 211 determines the types of the fixing jig and the assisting tool based on the imaging direction included in the past imaging information related to the examination information as the determination target, as the recommended types of the fixing jig and the assisting tool. Specifically, the controller 211 determines the types of the fixing jig and the assisting tool used in the past radiographic imaging performed in the same direction as the imaging direction of this time, as the recommended types of the fixing jig and the assisting tool.

The controller 211 determines the types of the fixing jig and the assisting tool based on the information about the imaging type included in the past imaging information related to the examination information as the determination target, as the recommended types of the fixing jig and the assisting tool. Specifically, the controller 211 determines the types of the fixing jig and the assisting tool used in the past radiographic imaging of the same type as the imaging type of this time, as the recommended types of the fixing jig and the assisting tool.

The controller 211 determines the types of the fixing jig and the assisting tool based on the irradiation conditions included in the past imaging information related to the examination information as the determination target, as the recommended types of the fixing jig and the assisting tool. Specifically, the controller 211 determines the types of the fixing jig and the assisting tool used in the past radiographic imaging under the same irradiation conditions as the irradiation conditions of this time, as the recommended types of the fixing jig and the assisting tool.

The controller 211 determines the types of the fixing jig and the assisting tool based on the precautions at the time of the past imaging included in the past imaging information related to the examination information as the determination target, as the recommended types of the fixing jig and the assisting tool.

The controller 211 determines the types of the fixing jig and the assisting tool based on the imaging location included in the past imaging information related to the examination information as the determination target, as the recommended types of the fixing jig and the assisting tool. Specifically, the controller 211 determines the types of fixing jig and assisting tool used in the past radiographic imaging at the same location as the visit location of this time, as the recommended types of fixing jig and assisting tool.

The controller 211 determines the types of the fixing jig and the assisting tool based on the patient information and the radiographer information related to the examination information as the determination target, as the recommended types of the fixing jig and the assisting tool. For example, the controller 211 determines the types of the fixing jig and the assisting tool according to the combination of the physique of the patient and the gender of the radiographer. Specifically, a case will be described in which the physique of the patient is larger than the predetermined threshold, the gender of the radiographer is female, and the power shortage of the radiographer is expected. In this case, the controller 211 determines that a fixing jig is required as the recommended type of fixing jig and assisting tool.

The controller 211 determines the types of the fixing jig and the assisting tool based on the information about the type of imaging included in the examination information as the determination target, as the recommended types of the fixing jig and the assisting tool. For example, if the information about the type of imaging is still imaging, the controller 211 determines a fixing jig for fixing the subject, as the recommended type of fixing jig and assisting tool. In a case where the information about the type of imaging is dynamic imaging, the controller 211 determines an assisting tool for assisting a portion of the subject to be moved and a fixing jig for fixing a portion of the subject not to be moved, as the recommended types of fixing jig and assisting tool.

The controller 211 determines the types of the fixing jig and the assisting tool based on the medical precautions related to the examination information as the determination target and the residence in the facility, as the recommended types of the fixing jig and the assisting tool. For example, a description will be given of a case where the medical precaution is a hunched posture and the residence in the facility is a ward using a bed that is incapable of reclining or the like. In this case, the controller 211 determines a cushion or the like to be put at the waist portion of the subject, which is used when the imaging posture is the sitting posture, as the type of the recommended fixing jig and assisting tool.

The controller 211 determines the types of the fixing jig and the assisting tool based on the pregnancy state of the patient related to the examination information as the determination target, as the recommended types of the fixing jig and the assisting tool. For example, in a case where the patient is pregnant and the posture that can be taken is limited, the controller 211 determines a cushion or the like to be put at the waist portion of the patient, as the type of the recommended fixing jig and assisting tool.

The controller 211 determines the types of the fixing jig and the assisting tool based on the age of the patient related to the examination information as the determination target, as the recommended types of the fixing jig and the assisting tool. For example, if the age of the patient is equal to or more than a predetermined threshold value, the controller 211 determines that the assisting tool is necessary as the type information of the recommended fixing jig and assisting tool.

The controller 211 determines the types of the fixing jig and the assisting tool based on the medical history of the patient related to the examination information as the determination target, as the recommended types of the fixing jig and the assisting tool. For example, the controller 211 determines, based on the medical history of the patient, whether the patient is bedridden, whether the patient has a tube, whether the patient has a posture which the patient cannot take, and the like. Next, the controller 211 determines the types of the fixing jig and the assisting tool according to the determination results, as the recommended types of the fixing jig and the assisting tool. Specifically, a case will be described in which the medical history of the patient is a fracture of the lung field ribs, and the patient cannot, by his/her own, take a posture for imaging of the side of the chest on the bed. In this case, the controller 211 determines that an assisting tool for supporting the patient is required as the recommended type of fixing jig and assisting tool.

The controller 211 determines whether or not the fixing jig and the assisting tool are necessary based on the imaging direction included in the examination information as the determination target and the height and weight of the patient related to the examination information. Next, the controller 211 determines the determination result as the type information of the recommended fixing jig and assisting tool.

The controller 211 determines, based on the statistical information, types of a fixing jig and an assisting tool that are frequently used in combination with the visit location included in the examination information as the determination target, the imaging information, and/or the radiographer information, as the recommended types of the fixing jig and the assisting tool.

The recommended equipment information of the present embodiment includes recommended support personnel information.

A specific method of determining the recommended support personnel information will be described below.

For example, the controller 211 determines, as the recommended support personnel information, the support personnel information based on the patient information and the radiographer information related to the examination information as the determination target. For example, the controller 211 determines it in accordance with a combination of the physique of the patient and the gender of the radiographer. Specifically, a case will be described in which the physique of the patient is larger than the predetermined threshold, the gender of the radiographer is female, and the power shortage of the radiographer is expected. In this case, the controller 211 determines that the support personnel is required as the recommended support personnel information.

The controller 211 may determine the recommended equipment information by combining the above-described determination methods.

The contents of the determination methods may be configured to be changeable for each user or facility.

The controller 211 may determine the recommended equipment information on the basis of the ON/OFF information of the power of the FPD 1, the association information (registration information) between the console 3 and the FPD 1, the information as to whether the FPD 1 is in use, and the like.

As described above, the controller 211 determines the recommended equipment information on the basis of the examination information on the subject to be radiographed by the medical cart RC. That is, the imaging equipment to be used by the photographer at the destination is determined based on the examination information on the subject to be subjected to radiographic imaging by the movable radiographic imaging apparatus.

The controller 211 determines the recommended equipment information on the basis of one piece of examination information on the subject to be subjected to radiographic imaging by the medical cart RC. That is, the imaging equipment to be used by the photographer at the destination is determined on the basis of one piece of examination information.

The controller 211 determines the recommended equipment information on the basis of a plurality of pieces of examination information on the subject to be subjected to radiographic imaging by the medical cart RC. That is, the imaging equipment to be used by the photographer at the destination is determined based on a plurality of pieces of examination information.

The controller 211 determines the recommended equipment information on the basis of the examination information on the subject to be radiographed by the medical cart RC and the related information related to the examination information. That is, the imaging equipment to be used by the photographer at the destination is determined on the basis of the examination information on the subject and the related information related to the examination information.

The controller 211 determines the recommended equipment information based on the examination information on the subject to be radiographed by the medical cart RC and the equipment state information. That is, the imaging equipment to be used by the photographer at the destination is determined on the basis of the examination information on the subject and the information on the state of the imaging equipment.

Next, the controller 211 displays the recommended equipment information determined in Step S3 on the display part 31 (Step S4), and ends this process. The step S4 is a notification step.

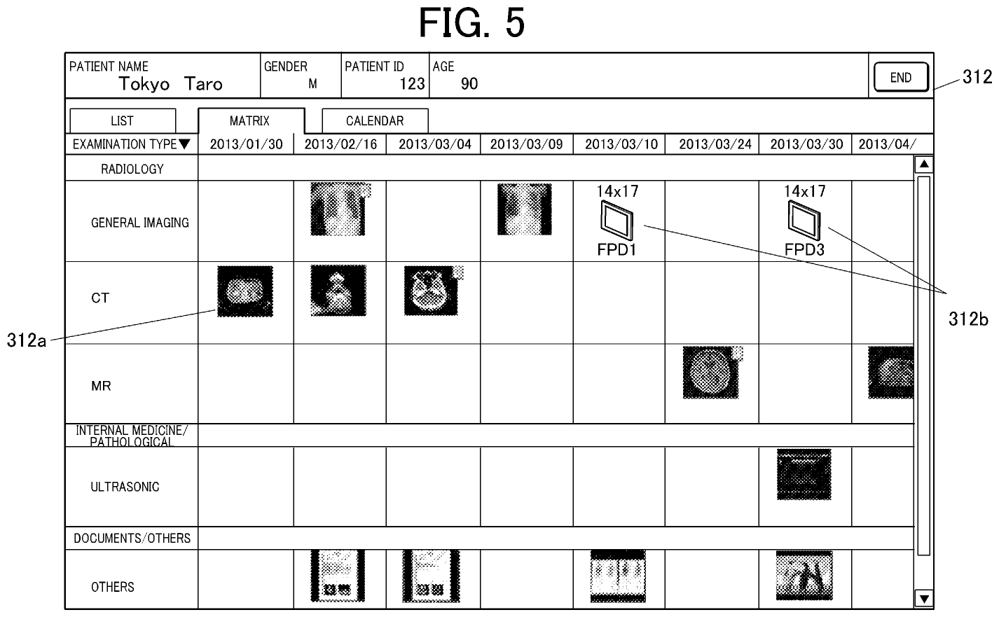

FIG. 5 shows a display screen 312 for displaying the recommended equipment information.

In the example illustrated in FIG. 5, the controller 211 collectively displays information on examinations in which one patient is imaged, on the display screen 312.

The vertical axis of the display screen 312 is the examination type, and the horizontal axis is the time axis. That is, the controller 211 displays, on the display screen 312, information on the examinations in a timeline format for each examination type.

The controller 211 may set the horizontal axis of the display screen 312 not in units of date illustrated in FIG. 5 but in units of time.

The controller 211 may display, on the display screen 312, not information on the examinations in which one patient is imaged but information on the examinations in which imaging is performed in one day collectively.

The controller 211 displays a thumbnail image of a radiographic image captured in each examination, such as the thumbnail image 312a, for the imaging-done examination.

The controller 211 displays an icon 312b indicating the recommended equipment determined in Step S3 for the examination of imaging to be performed.

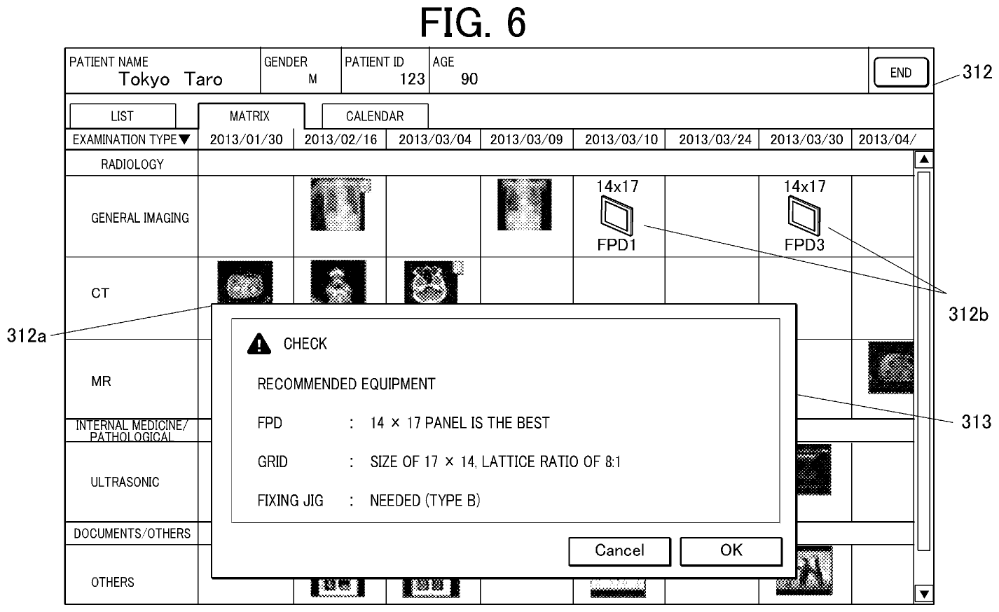

When receiving a click input of the icon 312b by the user via the operation part 32, the controller 211 displays a recommended equipment information screen 313 illustrated in FIG. 6 on the display screen 312.

In the example illustrated in FIG. 6, the controller 211 displays, on the recommended equipment information screen 313, the size of the FPD 1, the type of the grid, and the necessity and type of the fixing jig that are recommended as the recommended equipment information.

The display of the recommended equipment information on the recommended equipment information screen 313 is not limited to the example illustrated in FIG. 6.

For example, a case in which the size of an FPD 1 included in examination information as the determination target does not match the size of an FPD 1 included in past imaging information related to the examination information will be described. In this case, as described above, the controller 211 determines, in Step S3, the size of the FPD 1 included in the past imaging information as the recommended size of the FPD 1.

In this case, the controller 211 may display, on the recommended equipment information screen 313, a reason why the size of the FPD 1 was used in the past imaging.

A case in which the resolution included in the examination information as the determination target does not match the resolution included in the past imaging information related to the examination information will be described. In this case, the controller 211 determines, in Step S3, the type of FPD 1 capable of performing at the resolutions included in the past imaging information, as the recommended type of FPD 1.

In this case, the controller 211 may display, on the recommended equipment information screen 313, a reason why the type of FPD 1 was used in the past imaging.

The controller 211 may display, on the recommended equipment information screen 313, precautions at the time of past imaging included in the past imaging information related to the examination information that is the determination target.

The controller 211 may display, on the recommended equipment information screen 313, an FPD 1, a fixing jig, and/or an assisting tool according to the precautions at the time of past imaging included in the past imaging information related to the examination information as the determination target.

When displaying the identification information of the recommended FPD 1 on the recommended equipment information screen 313, the controller 211 may display the following thereon.

To be specific, the controller 211 may further display location information of the recommended FPD 1.

If the remaining battery level of a recommended FPD 1 is equal to or lower than a predetermined threshold value, the controller 211 may further display information on the remaining battery level of the FPD 1.

In a case where the recommended support personnel information is displayed as the recommended equipment information on the recommended equipment information screen 313, the controller 211 may display the following.

For example, a case will be described in which the physique of the patient is larger than the predetermined threshold, the gender of the radiographer is female, and the power shortage of the radiographer is expected. In this case, the controller 211 may display a suggestion to perform imaging by two radiographers.

In the example illustrated in FIG. 6, the controller 211 displays one size of the FPD 1, one type of the grid and one type of the fixing jig that are recommended, as the recommended equipment information, but is not limited thereto. The controller 211 may display a plurality of types of each of the respective pieces of the imaging equipment recommended as the recommended equipment information. In a case where the controller 211 displays a plurality of types of each piece of recommended imaging equipment, it is preferable to display the types together with the order of recommendation.

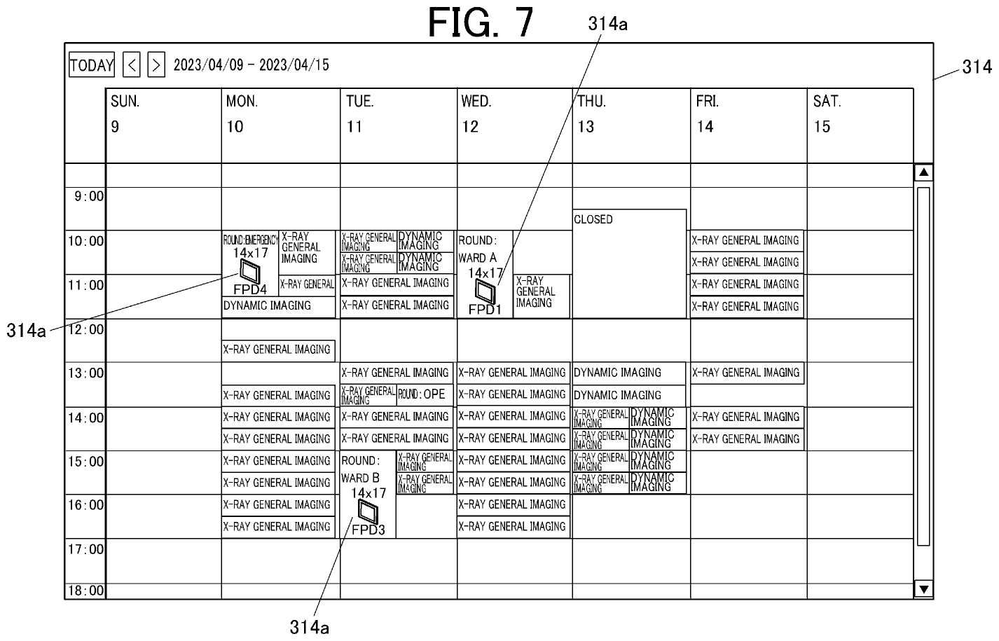

FIG. 7 illustrates a display screen 314 which is another example of displaying the recommended equipment information.

In the example illustrated in FIG. 7, the vertical axis of the display screen 314 is the time axis, and the horizontal axis is the date. The controller 211 displays information on examinations in a timeline format on the display screen 314.

The controller 211 displays an icon 314a indicating the recommended equipment determined in Step S3 for the examination of imaging to be performed.

When receiving a click input of the icon 314a by the user via the operation part 32, the controller 211 displays a recommended equipment information screen similar to the recommended equipment information screen 313 illustrated in FIG. 6, on the display screen 314.

That is, as illustrated in FIGS. 5 to 7, the controller 211 serving as the notification section performs notification by displaying, based on a predetermined operation by the user (person who uses or a using person), information (recommended equipment information) about the imaging equipment to be used by the user (photographer) at the destination. The predetermined operation in this case is a click input to the icon 312b/314a indicating the imaging equipment by the using person.

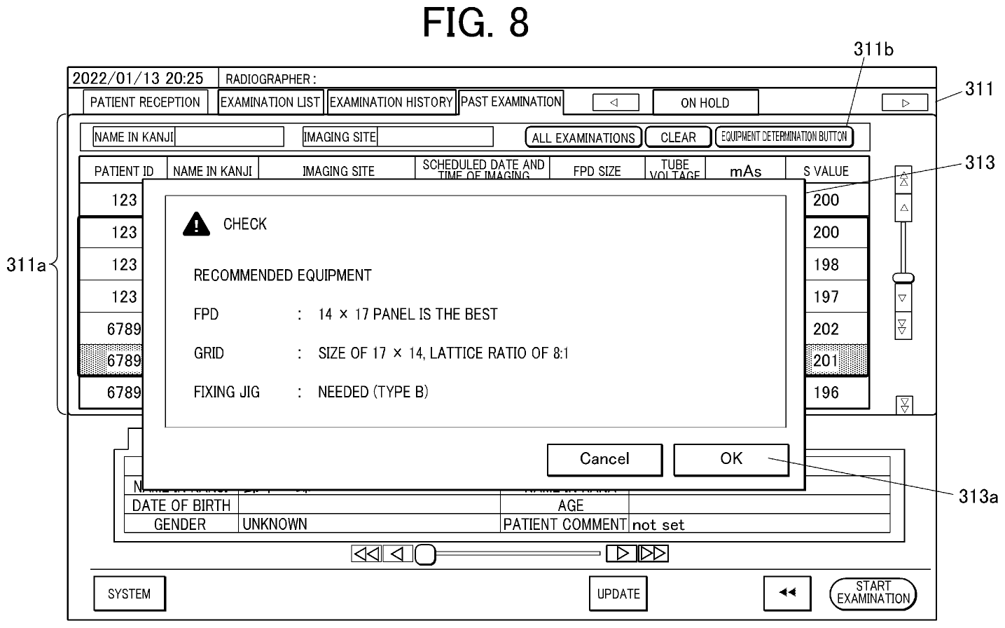

FIG. 8 shows another example of displaying the recommended equipment information.

The example illustrated in FIG. 8 is a case where, in Step S2, the controller 211 receives the user's selections of a plurality of pieces of examination information as the determination target from among the pieces of examination information displayed in the list 311a.

In this case, in Step S3, upon receiving a click input on the equipment determination button 311b after the selection operations by the user, the controller 211 determines recommended equipment information corresponding to the selected plurality of pieces of examination information.

In Step S4, as shown in FIG. 8, the controller 211 displays a recommended equipment information screen 313 for displaying the recommended equipment information determined in Step S3, on the display screen 311.

That is, the controller 211 as the notification section performs notification by displaying the information (recommended equipment information) on the imaging equipment to be used by the user (photographer) at the destination, based on a predetermined operation by the user (person who uses or a using person).

The predetermined operation in this case is specification of an examination of a radiographic imaging target by the using person. The specification of the examination of the radiographic imaging target by the using person is selection of the examination information as the determination target by the user.

The type, the display format, and the expression of the recommended equipment information that is displayed in Step S4 may be configured to be settable/changeable for each facility.

When receiving a click input of an OK button 313a (see FIG. 8) provided on the recommended equipment information screen 313 by the user, the controller 211 performs the following process(es). To be specific, the controller 211 changes the size of the FPD 1 included in the examination information selected as the determination target to the size of the FPD 1 as the recommended equipment information.

In the example illustrated in FIG. 8, in a case where the controller 211 receives a click input of the equipment determination button 311b by the user, Steps S3 and S4 are performed, but this is not a limitation. The controller 211 may perform Steps S3 and S4 in a case where a pressing operation of an examination start button (not illustrated) by the user is received. The examination start button is provided, for example, on the generator main body 21.

A case in which the controller 211 selects all pieces of examination information displayed in the list 311a in Step S2 as the determination target will be described.

In this case, the controller 211 performs Steps S3 and S4 without receiving a user's operation of selecting a piece of examination information.

In Step S4, the controller 211 provides an area for displaying the recommended equipment information determined in Step S3 on the display screen 311, and displays the recommended equipment information.

That is, the controller 211 as the notification section performs notification by displaying the information (recommended equipment information) on the imaging equipment to be used by the user (photographer) at the destination, based on a predetermined operation by the user (person who uses or a using person). The predetermined operation in this case is an instruction to update the examination information by the using person in Step S1, or input of patient information that is related information related to the examination information by the using person in Step S1.

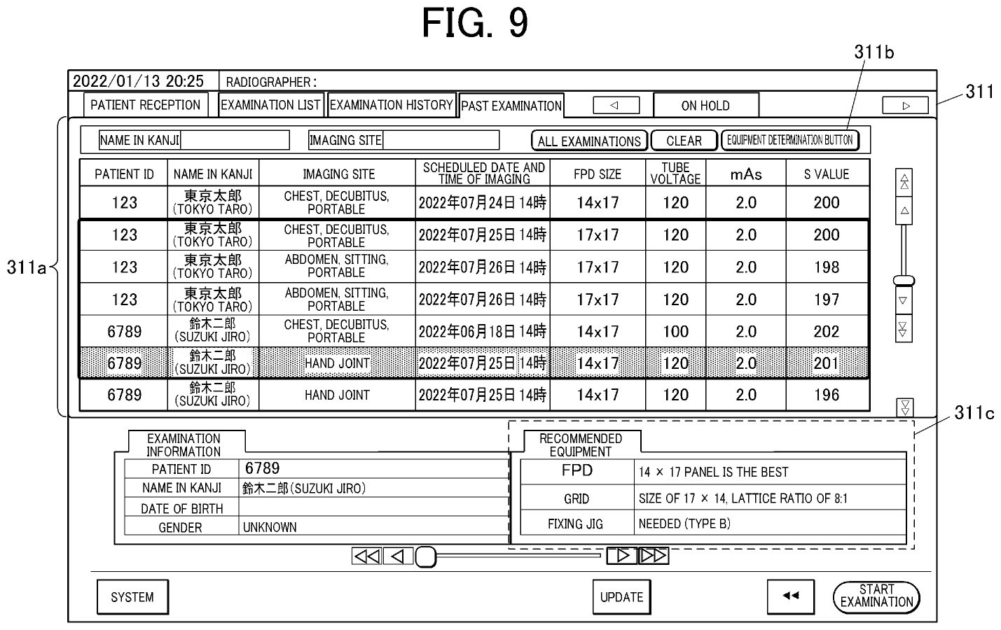

FIG. 9 shows another example of displaying the recommended equipment information.

In the example shown in FIG. 9, similarly to the example shown in FIG. 8, in Step S2, the controller 211 receives the user's selections of a plurality of pieces of examination information as the determination target from among the pieces of examination information displayed in the list 311a.

In Step S4, as illustrated in FIG. 9, the controller 211 displays the recommended equipment information determined in Step S3 in the display area 311c of the display screen 311.

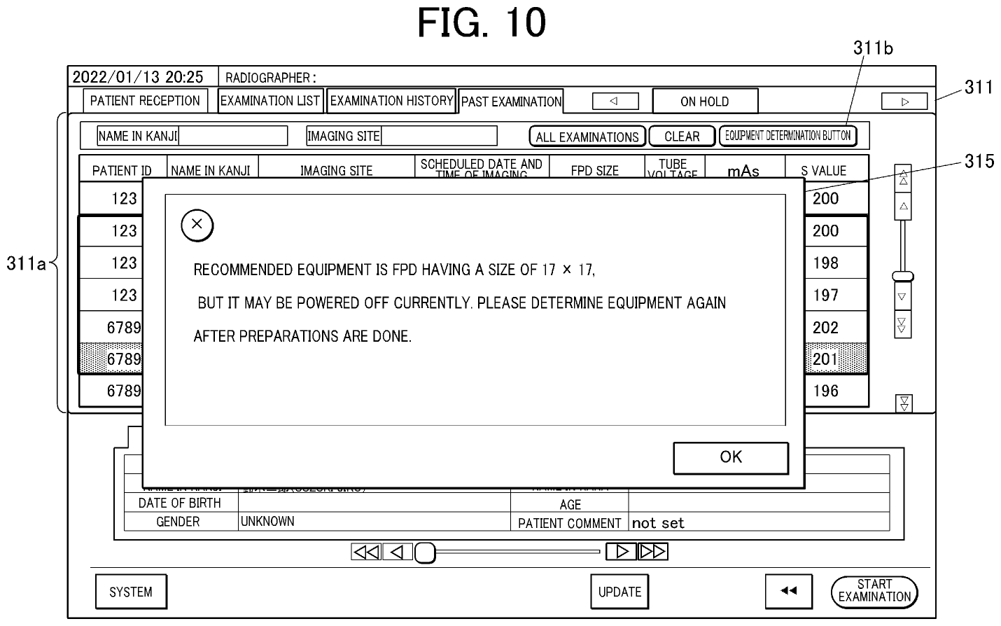

FIG. 10 shows another example of displaying the recommended equipment information.

The example illustrated in FIG. 10 is a display example in a case where the recommended equipment determined by the controller 211 in Step S3 may not be able to be used. In this case, the controller 211 displays, in Step S4, a caution screen 315 on the display screen 311.

For example, a case will be described in which, in Step S3, the controller 211 determines the recommended size of FPD 1 to be “17×17”, but the power of the FPD 1 of the size is currently off. In this case, the controller 211 displays a message indicating that on the caution screen 315. The controller 211 displays, on the caution screen 315, a message prompting the user to resolve the cause of unavailability of the recommended equipment. In the example illustrated in FIG. 10, the controller 211 displays, as a message prompting to turn on the power of the recommended FPD 1, a message “Please determine equipment again after preparations”.

The controller 211 may cause the display form of the caution screen 315 to be different from that of the recommended equipment information screen 313. The controller 211, for example, changes the color or font of the caution screen 315 from that of the recommended equipment information screen 313, or adds a mark or the like to the caution screen 315 for highlighted display.

Thus, the user can easily recognize that the recommended equipment may not be usable.

The controller 211 may display the caution screen 315 in Step S4 in the following case.

To be specific, this is a case where the recommended FPD 1 determined in Step S3 is not associated with the console 3. In other words, this is a case where the recommended FPD 1 is not registered in the console 3. In this case, the controller 211 may display the information of the FPD 1 that is the second in the order of recommendation, on the caution screen 315.

Alternatively, it is a case where the recommended FPD 1 determined in Step S3 and the FPD stored in the FPD storage section 26 of the radiation generation apparatus 2 do not match.

That is, the controller 211 serving as the notification section notifies the using person when the FPD stored in the FPD storage section 26 does not match the FPD determined as the imaging equipment to be used by the photographer at the destination.

The controller 211 may display the caution screen 315 in Step S4 in the following case.

To be specific, this is a case where the remaining battery level of the recommended FPD 1 determined in Step S3 is equal to or lower than a predetermined threshold value.

Alternatively, it is a case where the size of the recommended FPD 1 determined in Step S3 is different from the size of the FPD 1 corresponding to the imaging included in the examination information as the determination target.

Alternatively, it is a case where the recommended FPD 1 determined in Step S3 is being used in another examination.

Alternatively, it is a case where the location of the recommended FPD 1 determined in Step S3 is a place apart from the medical cart RC, which is the own apparatus, by a predetermined distance or more.

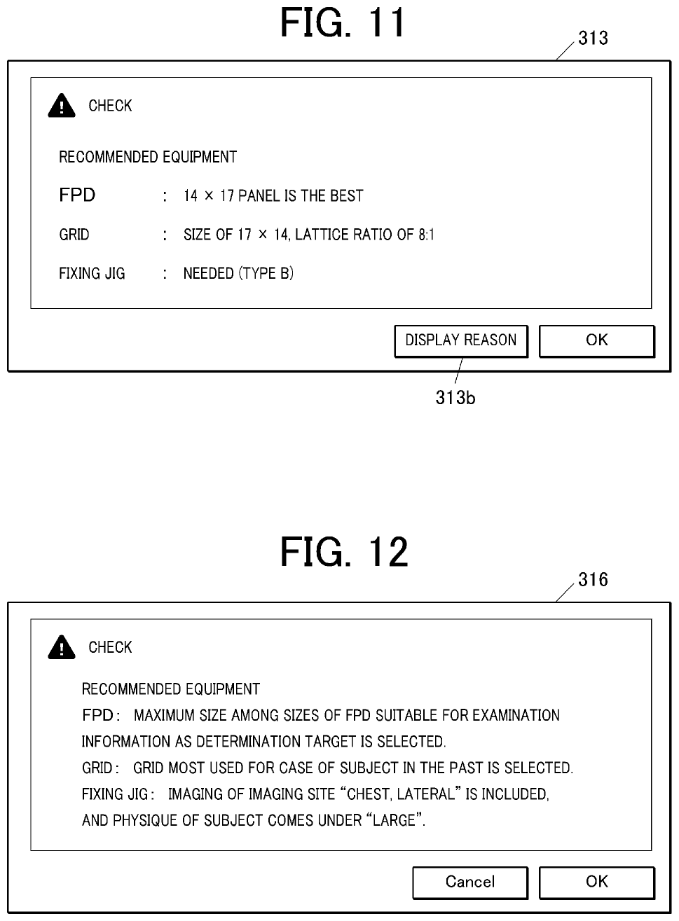

FIG. 11 shows another example of displaying the recommended equipment information.

As illustrated in FIG. 11, the controller 211 may provide a reason display button 313b on the recommended equipment information screen 313 for displaying the recommended equipment information.

When receiving a click input of the reason display button 313b by the user via the operation part 32, the controller 211 displays a reason screen 316 illustrated in FIG. 12.

The controller 211 displays, on the reason screen 316, the determination reason why the recommended equipment information has been determined in Step S3 as a recommendation reason.

By displaying the recommendation reason, it is possible to more convince and educate the user such as the radiographer.

The controller 211 may provide the recommended equipment information screen 313 with scroll and display the recommendation reason under the recommended equipment information.

The controller 211 may display the recommendation reason for each piece of imaging equipment or may collectively display the recommendation reasons for pieces of imaging equipment.

The controller 211 may display imaging equipment that is another candidate of the recommended equipment together with the recommendation reason.

The controller 211 may display the essential reason for recommendation in a color or a font different from that of the other reasons for recommendation, or may perform highlighted display by adding a mark or the like. The essential reason for recommendation will be described below. For example, if the examination information as the determination target includes information indicating that the imaging is dynamic imaging, the controller 211 determines an FPD 1 definitely/always capable of dynamic imaging as a recommended FPD 1. In this case, the controller 211 displays, as an essential recommendation reason, a message indicating that an FPD 1 capable of dynamic imaging has been selected because the imaging is dynamic imaging.

After execution of the recommended equipment information display process, the user such as the radiographer goes from the standby place to the visit location where the subject to be imaged is present, together with the imaging equipment indicated by the recommended equipment information displayed in the above process. Next, the user starts radiographic imaging of the subject using the imaging equipment.

### 3. Others

Although the present invention has been described based on the above embodiment, the descriptions in the above embodiment are some of preferred examples of the radiographic imaging system according to the present invention, and the present invention is not limited thereto.

For example, in Step S4 of the recommended equipment information display process, the controller 211 may display the recommended equipment information for each of predetermined units. The predetermined units are units of imaging, units of examination, units of round visit, units of ward, units of time frame of a schedule, or the like.

If the recommended equipment information is displayed in units of imaging, the controller 211 may display the recommended equipment information in the form of a list.

The controller 211 may display the recommended equipment information in the form of symbols or perform notification through sound by the sound output section 33, in addition to or instead of display in the form of letters, icons, or the like.

In Step S4 of the recommended equipment information display process, the controller 211 may transmit the recommended equipment information determined in Step S3 to another apparatus via the communication part 214. The other apparatus is a console included in a radiographic imaging system for general imaging, another medical cart RC, or the like. Upon receiving the recommended equipment information, the other apparatus displays the recommended equipment information on a display part thereof.

In this case, the controller 211 may transmit, to the other apparatus, only the recommended equipment information corresponding to the examination to be performed by the other apparatus.

The recommended imaging equipment may be determined as follows.

A case in which the controller 211 displays, for example, a plurality of recommended FPDs in Step S4 of the recommended equipment information display process will be described.

In this case, the user such as the radiographer carries a plurality of recommended FPDs to the visit location. Next, the user causes the controller 211 to perform the recommended equipment information display process again at the visit location, and in Step S1, inputs additional information based on the situation of the visit location. Next, based on the recommended equipment information displayed in Step S4, the user determines an FPD to be used for imaging from among the plurality of FPDs that the user has brought.

### 4. Modification Example

Next, a modification example of the present invention will be described.

The radiographic imaging support apparatus of the present invention may be applied to a console in a radiographic imaging system for general imaging.

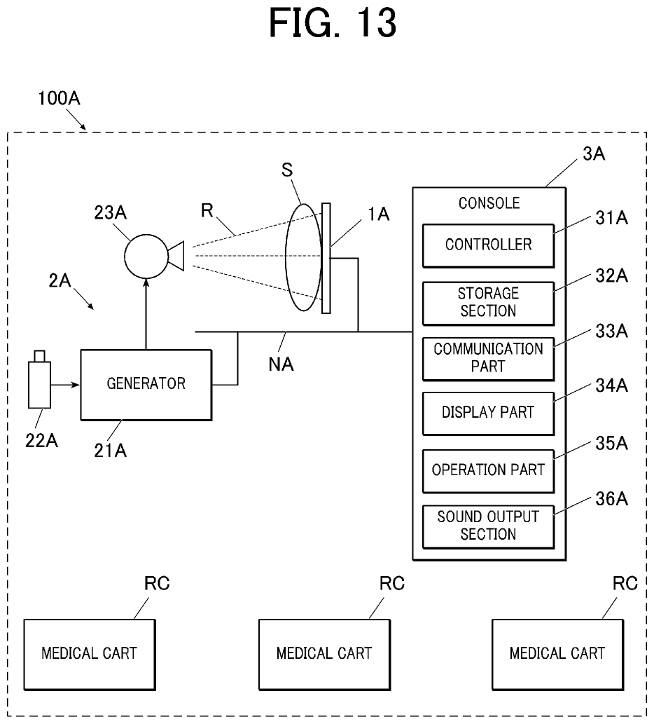

FIG. 13 illustrates a schematic configuration diagram of a radiographic imaging system 100A for general imaging of the present modification example.

As illustrated in FIG. 13, the radiographic imaging system 100A includes a radiographic imaging apparatus 1A, a radiation generation apparatus 2A, a console 3A, and a medical cart RC.

The radiographic imaging apparatus 1A, the radiation generation apparatus 2A, and the console 3A can communicate with each other via, for example, a communication network NA.

The radiographic imaging apparatus 1A generates radiographic image data corresponding to radiation R emitted from the radiation generation apparatus 2A.

The radiation generation apparatus 2A includes a generator 21A, an irradiation instruction switch 22A, and a tube 23A.

The generator 21A applies a voltage corresponding to preset imaging conditions to the tube 23A on the basis of an operation on the irradiation instruction switch 22A.

When the voltage is applied from the generator 21A, the tube 23A generates the radiation R (for example, X-rays or the like) of a dose corresponding to the applied voltage.

The console 3A can set imaging conditions in at least one of the radiographic imaging apparatus 1A and the radiation generation apparatus 2A.

The console 3A can acquire the radiographic image data generated by the radiographic imaging apparatus 1A, and store the radiographic image data in itself and/or transmit the radiographic image data to another external apparatus (PACS, etc.).

The console 3A includes a controller 31A (hardware processor), a storage section 32A (storage), a communication part 33A, a display part 34A, an operation part 35A, and a sound output section 36A.

The storage section 32A, the communication part 33A, the display part 34A, the operation part 35A, and the sound output section 36A have the same configurations as the storage section 212, the communication part 214, the display part 31, the operation part 32, and the sound output section 33, respectively.

The controller 31A includes a CPU and a RAM. The CPU reads various programs stored in the storage section 32A and loads the programs into the RAM. The CPU executes various kinds of processing in cooperation with the program, and controls each component of the console 3A.

In the present modification example, a case where the console 3A, which is a console for general imaging, performs the recommended equipment information display process will be described. In this case, the controller 31A performs notification of information on the imaging equipment to be used by the radiographer at the destination, the information being determined based on the examination information on the subject to be radiographed by the medical cart RC as the movable radiographic imaging apparatus. The controller 31A functions as the notification section. In this case, the console 3A is the radiographic imaging support apparatus. The imaging equipment in this case includes the medical cart RC that performs round-visit imaging.

In Step S3 of the recommended equipment information display process, the controller 31A determines the recommended equipment information corresponding to the examination information as the determination target selected in Step S2, as in the above embodiment. The controller 31A determines the recommended equipment information on the basis of the examination information, the related information, and the equipment state information.

The recommended equipment information of the present modification example includes type information of a recommended medical cart RC.

The type information of the recommended medical cart RC is preferably notified before the user such as the radiographer selects the medical cart RC at the standby place. Therefore, in this case, the controller 31A of the console 3A for general imaging performs the recommended equipment information display process. A method by which the controller 31A determines the type of the recommended medical cart RC will be described below.

For example, the controller 31A determines the type of the medical cart RC based on the information on the type of imaging included in the examination information as the determination target, as the recommended type of the medical cart RC. For example, if the information on the type of imaging indicates dynamic imaging, the controller 31A determines the type of the medical cart RC capable of dynamic imaging as the recommended type of the medical cart RC.

The recommended equipment information of the present modification example includes identification information of a recommended medical cart RC.

The identification information of the recommended medical cart RC is preferably notified before the user such as the radiographer selects the medical cart RC at the standby place. Therefore, in this case, the controller 31A of the console 3A for general imaging performs the recommended equipment information display process. A method by which the controller 31A determines the identification information of the recommended medical cart RC will be described below.

For example, the controller 31A determines, as the recommended medical cart RC, a medical cart RC based on the patient information related to the examination information as the determination target and the equipment state information. For example, the controller 31A determines the medical cart RC whose location is the in-facility residence of the subject, as the recommended medical cart RC.

The controller 31A determines, as the recommended medical cart RC, a medical cart RC based on the visit location included in the examination information as the determination target and the equipment state information. For example, the controller 31A determines a medical cart RC whose location is relatively close to the visit location as the recommended medical cart RC.

The controller 31A determines to exclude, from the recommended medical carts RC, a medical cart RC whose location included in the equipment state information is separated by a predetermined threshold or more from the visit location included in the examination information as the determination target.

The controller 31A determines to exclude a medical cart RC whose state of charge included in the equipment state information is equal to or less than a predetermined threshold from the recommended medical carts RC.

The controller 31A determines to exclude, from the recommended medical carts RC, a medical cart RC whose amount of charge included in the equipment state information is less than the amount of charge with which imaging can be performed included in the examination information as the determination target.

Next, in Step S4 of the recommended equipment information display process, as in the above embodiment, the controller 31A displays the recommended equipment information determined in Step S3 on the display part 34A.

On the recommended equipment information screen 313 displayed on the display part 34A, the controller 31A may display the following when displaying the identification information of the recommended medical cart RC.

Specifically, the controller 31A may display the location information of the recommended medical cart RC.

If the state of charge of the recommended medical cart RC is equal to or less than a predetermined threshold, the controller 31A may display the charge state information of the medical cart RC.

The controller 31A may display the caution screen 315 on the display part 34A in Step S4 in the following cases. Specifically, this is a case where the recommended medical cart RC determined in Step S3 is being used in another examination.

In Step S4, the controller 31A may transmit the recommended equipment information determined in Step S3 to the console 3 of the medical cart RC. When receiving the recommended equipment information, the controller 211 of the console 3 displays the recommended equipment information on the display part 31. In this case, the controller 211 functions as the notification section.

In the embodiment and the modification example described above, an external apparatus such as the RIS 40 or a terminal apparatus exemplified by a PC used by the user may perform the recommended equipment information display process. In this case, the external apparatus may determine the recommended equipment information and transmit the determined recommended equipment information to the console 3 of the medical cart RC.

The display items and display form of the recommended equipment information may be set for each apparatus that displays the recommended equipment information. For example, the console 3A of the radiographic imaging system 100A for general imaging displays information on a medical cart as the recommended equipment information. Meanwhile, the medical cart RC does not display information on the medical cart RC as the recommended equipment information.

### 5. Advantageous Effects

As described above, according to the present embodiment, the console 3 or 3A as the radiographic imaging support apparatus includes the notification section (the controller 211 or 31A) to perform notification of the information on the imaging equipment to be used by the photographer at the destination, the imaging equipment being determined based on the examination information on the subject who undergoes radiographic imaging by the movable radiographic imaging apparatus (the medical cart RC).

Therefore, the user can suitably prepare imaging equipment suitable for imaging at the visit location before going to the visit location.

Thus, imaging equipment necessary for imaging can be more reliably taken, and imaging can be efficiently performed at the visit location. In addition, it is possible to reduce a burden on the user.

In the radiographic imaging support apparatus of the present embodiment, the imaging equipment is at least one of the radiographic imaging apparatus (FPD 1), a grid attached to and detached from the radiographic imaging apparatus, a jig (fixing jig) for fixing a subject, an assisting tool, and a movable radiographic imaging apparatus (medical cart RC).

Therefore, the user can suitably prepare an FPD 1, a grid, a fixing jig, an assisting tool, a medical cart RC, and/or the like suitable for imaging at the visit location before going to the visit location.

In the radiographic imaging support apparatus of the present embodiment, the examination information includes information on the type of radiographic imaging.

Therefore, the user can suitably prepare imaging equipment suitable for the type of radiographic imaging before going to the visit location.

In the radiographic imaging support apparatus of the present embodiment, the examination information is examination information on a plurality of subjects.

Therefore, the user can suitably prepare imaging equipment suitable for examinations of a plurality of subjects before going to the visit location. This makes it possible to reduce the number of pieces of imaging equipment to be taken to the visit location.

In the radiographic imaging support apparatus of the present embodiment, the imaging equipment is determined based on one piece of examination information.

Therefore, the user can appropriately prepare imaging equipment appropriate for one examination before going to the visit location.

In the radiographic imaging support apparatus of the present embodiment, the imaging equipment is determined based on a plurality of pieces of examination information.

Therefore, the user can appropriately prepare imaging equipment appropriate for a plurality of examinations before going to the visit location. This makes it possible to reduce the number of pieces of imaging equipment to be taken to the visit location.

In the radiographic imaging support apparatus of the present embodiment, the imaging equipment is determined on the basis of the examination information and the related information related to the examination information.

Therefore, the user can appropriately prepare imaging equipment appropriate for the examination information and the related information before going to the visit location.

In the radiographic imaging support apparatus according to the present embodiment, the related information is at least one of the condition of the subject, the age of the subject, the weight of the subject, the medical precautions about the subject, the medical history of the subject, the in-facility resistance of the subject, and the past radiographic imaging information.

Therefore, the user can preferably prepare the imaging equipment suitable for the examination information, the condition, the age, the weight, the medical precautions, the medical history and the residence in the facility of the subject and the past radiographic imaging information before going to the visit location.

In the radiographic imaging support apparatus of the present embodiment, the imaging equipment is determined based on the examination information and the information on the state of the imaging equipment (equipment state information).

Therefore, the user can appropriately prepare imaging equipment appropriate for the examination information and the equipment state information before going to the visit location.

In the radiographic imaging support apparatus according to the present embodiment, the notification section (the controller 211 or the controller 31A) notifies the information on the imaging equipment based on a predetermined operation performed by the using person.

Therefore, the user can be notified of imaging equipment suitable for imaging at the visit location at the timing when the user performs the predetermined operation.

In the radiographic imaging support apparatus of the present embodiment, the predetermined operation is one of specification by the using person of an examination for the radiographic imaging target, an instruction by the using person to update the examination information, an input by the using person of the related information related to the examination information, and a click input by the using person onto an icon indicating the imaging equipment.

Therefore, the user can be notified of the imaging equipment suitable for imaging at the visit location at any of the timing when the user specifies the examination of the radiographic imaging target, the timing when the user makes an instruct to update the examination information, the timing when the user inputs the patient information that is the related information, and the timing when the user clicks the icon indicating the imaging equipment.

In the radiographic imaging support apparatus according to the present embodiment, the notification section (controller 211, controller 31A) performs notification if the radiographic imaging apparatus (FPD 1) stored in the storage section (FPD storage section 26) of the movable radiographic imaging apparatus (medical cart RC) and the radiographic imaging apparatus (FPD 1) determined as the imaging equipment to be used by the photographer at the destination do not match.

Accordingly, the user can be prompted to store the FPD 1 determined as the imaging equipment in the FPD storage section 26.

Although the present invention has been described based on the above embodiment or the like, the descriptions in the above embodiment are some of preferred examples of the radiographic imaging system 100/100A according to the present invention, and the present invention is not limited thereto.

For example, in the above embodiment, the movable radiographic imaging apparatus is the medical cart RC, but the movable radiographic imaging apparatus may be a fluoroscopy apparatus.

In the above embodiment, the imaging equipment is at least one of the FPD 1, the grid attached to and detached from the FPD 1, the fixing jig for fixing the subject, the assisting tool, and the medical cart RC, but is not limited thereto. The imaging equipment may be a disinfection set or a radiation protection suit. The recommended equipment information may include necessity or unnecessity of the disinfection set or the radiation protection suit.

Although, in the above, an example in which the storage section 212/32A (semiconductor memory or HDD) is used as a computer-readable medium for the program according to the present invention has been disclosed, the present invention is not limited to this example. As another computer-readable medium, a portable recording medium such as a CD-ROM is applicable. Further, as a medium for providing data of the program according to the present invention via a communication line, a carrier wave is also applied to the present invention.

Detailed configurations and detailed operations of the respective components constituting the radiographic imaging system 100/100A in the embodiment/modification example can be appropriately changed without departing from the scope of the present invention.

Although embodiments of the present invention have been described and illustrated in detail, the disclosed embodiments are made for purposes of illustration and example only and not limitation. The scope of the present invention should be interpreted by terms of the appended claims.

The entire disclosure of Japanese Patent Application No. 2023-041460, filed on Mar. 16, 2023, including description, claims, drawings and abstract is incorporated herein by reference.

What is claimed is:

1. A radiographic imaging support apparatus comprising a hardware processor that performs notification of information on imaging equipment to be used by a photographer at a destination, the imaging equipment being determined based on examination information on a subject to be subjected to radiographic imaging by a movable radiographic imaging apparatus.

2. The radiographic imaging support apparatus according to claim 1, wherein the imaging equipment is at least one of a radiographic imaging apparatus, a grid that is detachably attached to the radiographic imaging apparatus, a jig for fixing the subject, an assisting tool and the movable radiographic imaging apparatus.

3. The radiographic imaging support apparatus according to claim 1, wherein the examination information includes information on a type of radiographic imaging.

4. The radiographic imaging support apparatus according to claim 1, wherein the examination information is examination information on a plurality of subjects.

5. The radiographic imaging support apparatus according to claim 1, wherein the imaging equipment is determined based on one piece of the examination information.

6. The radiographic imaging support apparatus according to claim 1, wherein the imaging equipment is determined based on a plurality of pieces of the examination information.

7. The radiographic imaging support apparatus according to claim 1, wherein the imaging equipment is determined based on the examination information and related information related to the examination information.

8. The radiographic imaging support apparatus according to claim 7, wherein the related information is at least one of a condition of the subject, an age of the subject, a weight of the subject, a medical precaution about the subject, a medical history of the subject, an in-facility residence of the subject, and past radiographic imaging information.

9. The radiographic imaging support apparatus according to claim 1, wherein the imaging equipment is determined based on the examination information and information on a state of the imaging equipment.

10. The radiographic imaging support apparatus according to claim 1, wherein the hardware processor performs the notification of the information on the imaging equipment based on a predetermined operation by a user.

11. The radiographic imaging support apparatus according to claim 10, wherein the predetermined operation is one of specification by the user of an examination for a radiographic imaging target, an instruction by the user to update the examination information, an input by the user of related information related to the examination information, and a click input by the user onto an icon indicating the imaging equipment.

12. The radiographic imaging support apparatus according to claim 1, wherein the hardware processor performs the notification in response to a radiographic imaging apparatus stored in a storage provided in the movable radiographic imaging apparatus not matching a radiographic imaging apparatus determined as the imaging equipment to be used by the photographer at the destination.

13. A radiographic imaging support method comprising performing notification of information on imaging equipment to be used by a photographer at a destination, the imaging equipment being determined based on examination information on a subject to be subjected to radiographic imaging by a movable radiographic imaging apparatus.

14. A non-transitory computer-readable recording medium storing a program that causes a hardware processor of a radiographic imaging support apparatus to perform notification of information on imaging equipment to be used by a photographer at a destination, the imaging equipment being determined based on examination information on a subject to be subjected to radiographic imaging by a movable radiographic imaging apparatus.
